(19) United States
(12) Patent Application Publication
(10) Pub. No.: US 2021/0295957 A1
(43) Pub. Date: Sep. 23, 2021

(54) METHOD AND SYSTEM FOR ASSESSING COACH QUALITY

(71) Applicant: Omada Health, Inc., San Francisco, CA (US)

(72) Inventors: Ryan Quan, San Francisco, CA (US); Stephen Hess, San Francisco, CA (US); Devin Ellsworth, San Francisco, CA (US); Luke Armistead, San Francisco, CA (US); Adrian James, San Francisco, CA (US)

(21) Appl. No.: 17/210,158

(22) Filed: Mar. 23, 2021

Related U.S. Application Data
(60) Provisional application No. 62/993,434, filed on Mar. 23, 2020.

(51) Int. Cl. G16H 10/20; G06N 7/00; G16H 50/30; G16H 20/00

(52) U.S. Cl. CPC G16H 10/20; G16H 20/00; G16H 50/30; G06N 7/005

(57) ABSTRACT

A method for assessing the quality of a coach includes collecting information; determining a set of metrics based on the information; and determining a coach quality based on the set of metrics. Additionally or alternatively, the method can include any or all of: determining a set of one or more outcomes key drivers (OKDs) associated with success of a participant and/or the health program; determining a set of models associated with the set of one or more OKDs; for each of the set of coaches, determining a baseline quality associated with the coach; producing an output and/or triggering an action based on the coach quality; and/or any other suitable processes.

### CROSS-REFERENCE TO RELATED APPLICATIONS

This application claims the benefit of U.S. Provisional Application No. 62/993,434, filed 23 Mar. 2020, which is incorporated in its entirety by this reference.

### TECHNICAL FIELD

This invention relates generally to the health field, and more specifically to a new and useful system and method for assessing quality of a set of coaches in the health field.

### BACKGROUND

Remote coaching platforms have been shown to have numerous benefits, such as being able to reach a large number of participants without geographical constraint, increasing the availability of a coach to a participant, and increasing the size of the coach workforce. Knowing and managing the quality of a set of remote coaches is challenging, however, and a large diversity in participant populations, along with a large coach workforce, can make this particularly difficult to assess.

Thus, there is a need in the remote coaching field to accurately assess the quality of a workforce of remote coaches.

### BRIEF DESCRIPTION OF THE FIGURES

FIG. 1 is a schematic of a system for assessing coach quality.

FIG. 2 is a schematic of a method for assessing coach quality.

FIG. 3 depicts a schematic of information flow within a variation of the method.

FIG. 4 depicts a variation of a coach dashboard.

FIG. 5 depicts a variation of a participant profile.

FIG. 6 depicts a schematic variation of information flow used in the method for assessing coach quality.

FIG. 7 depicts a variation of OKD metric values used to assess coach quality.

FIG. 8 depicts an example of multiple OKD metric values used to assess coach quality.

FIG. 9 depicts a variation of clustering of participant information used in a hierarchical model.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Overview

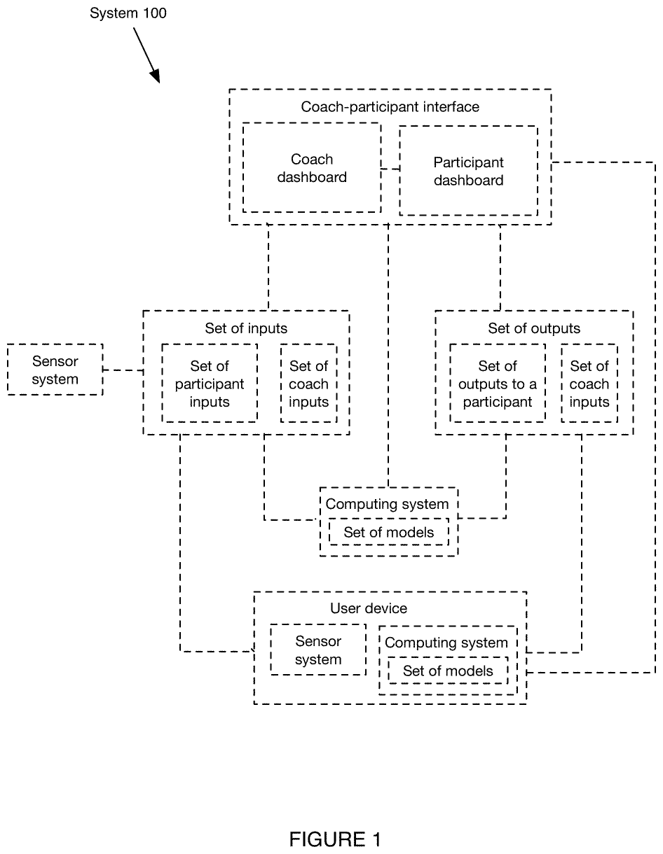

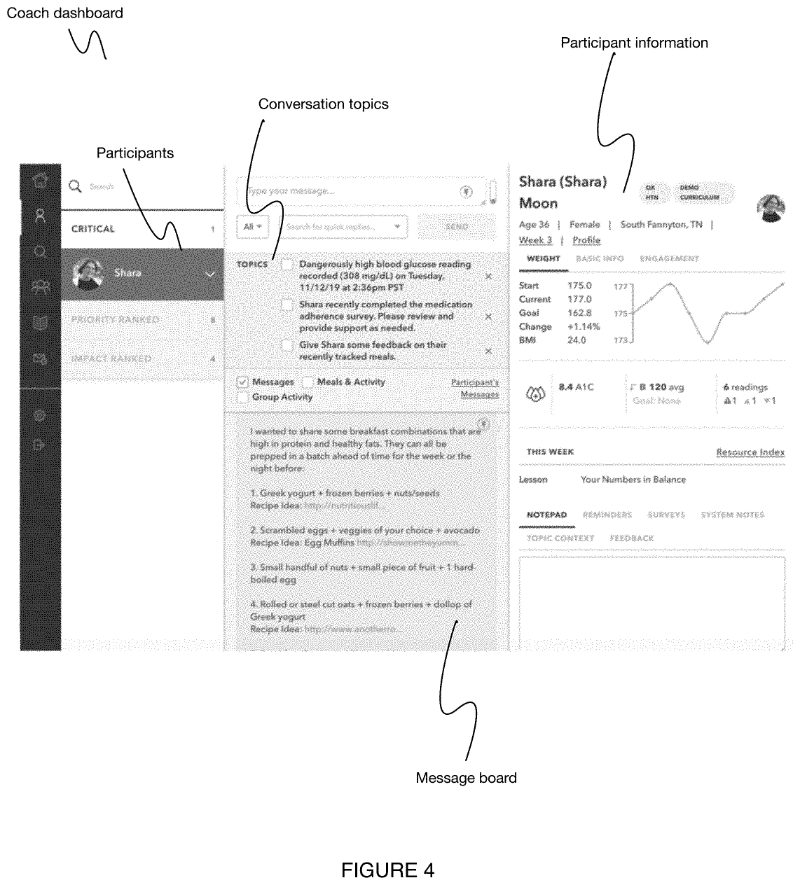

As shown in FIG. 1, a system 100 for assessing the quality of a coach can include and/or interface with any or all of: a computing system, a coach-participant interface including a coach dashboard (e.g., as shown in FIG. 4) and optionally a participant dashboard; a set of inputs (e.g., from a participant, from a coach, etc.); a set of outputs (e.g., to a participant, to a coach, etc.); a set of models (e.g., to convert the set of inputs into the set of outputs); a user device (e.g., to receive the set of inputs, to display the set of outputs, to display the dashboard, etc.); a sensor system (e.g., weight scale, step counter, accelerometer, etc.) to collect a set of one or more inputs from a set of participants; and/or any other suitable components.

Further additionally or alternatively, the system can include and/or interface with any or all of the components as described in any or all of: U.S. application Ser. No. 15/667,218, filed 2 Aug. 2017, and U.S. application Ser. No. 17/195,156, filed 8 Mar. 2021, each of which is incorporated herein in its entirety by this reference.

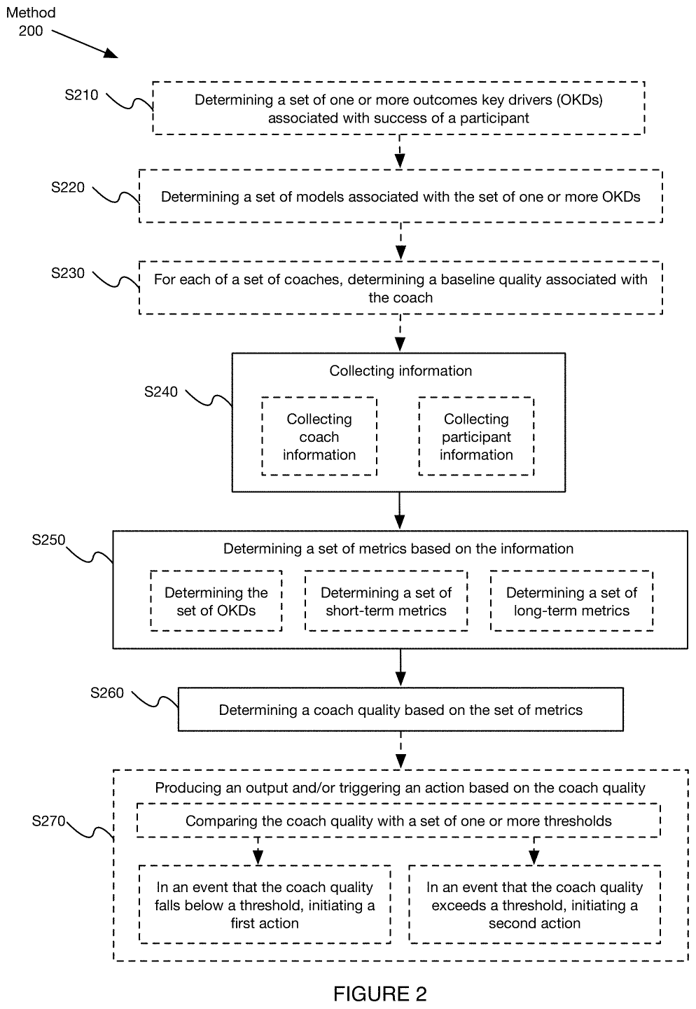

As shown in FIG. 2, a method 200 for assessing the quality of a coach includes collecting information S240; determining a set of metrics based on the information S250; and determining a coach quality based on the set of metrics S260. Additionally or alternatively, the method 200 can include any or all of: determining a set of one or more outcomes key drivers (OKDs) associated with success of a participant and/or the health program S210; determining a set of models associated with the set of one or more OKDs S220; for each of the set of coaches, determining a baseline quality associated with the coach S230; producing an output and/or triggering an action based on the coach quality S270; and/or any other suitable processes.

Further additionally or alternatively, the method 200 can include any or all of the processes described in any or all of: U.S. application Ser. No. 15/667,218, filed 2 Aug. 2017, and U.S. application Ser. No. 17/195,156, filed 8 Mar. 2021, each of which is incorporated in its entirety by this reference. The method 200 can be performed with a system as described above and/or any other suitable system.

### 2. Benefits

The system and method for assessing a coach quality can confer several benefits over current systems and methods.

In a first set of variations, the system and/or method confers the benefit of fairly assessing the quality of a coach in achieving one or more participant outcomes based only on factors that are within the coach's control. In specific examples, for instance, the system and/or method assess coach quality at least in part based on a set of adjusted metrics, wherein the adjusted metrics are adjusted to weed out various factors which can contribute (e.g., negatively, positively, etc.) to user outcomes that are outside of the coach's control. In looking at the coach's impact on helping a participant achieve a goal of weight loss, for instance, an adjusted weight loss can be used to determine the coach's quality in doing so, wherein factors which might affect the participant's ability to lose weight (e.g., existing conditions such as metabolic conditions, income level, age, gender, geographic proximity to healthy food, etc.) can be taken into account and not used to unfairly penalize (or unfairly overestimate) the coach for factors outside of his or her control.

In a second set of variations, additional or alternative to the first, the system and/or method confers the benefit of enabling a fully or nearly fully automated assessment of a set of coaches. Additionally or alternatively, the system and/or method can confer the benefit of fully or nearly fully automating the initiating a set of actions resulting from the assessment of coach quality. In preferred examples of this variation, all of this is achieved remotely from the coaches.

In a third set of variations, additional or alternative to those described above, the system and/or method confers the benefit of determining a widely applicable set of metrics reflective of patient progress, which can be used to assess the quality of a coach in effecting patient progress with respect to the metric(s). In specific examples, this prevents the need to micromanage a set of coaches, as this way for measuring quality does not require vigilant monitoring of each of the coach's interactions with his or her participants.

In a third set of variations, additional or alternative to those described above, the system and/or method confers the benefit of enabling the scaling of a coach workforce through an efficient (e.g., automated) and remote assessment and monitoring of coach quality. In a set of specific examples, for instance, a manager in charge of monitoring coaches can be automatically alerted and/or provided information with which to triage his or her management of a large set of coaches, such that the manager can be informed of which coaches are performing well and which coaches are struggling.

In a fourth set of variations, additional or alternative to those described above, the system and/or method confers the benefit of enabling results-based compensation in the health space. In specific examples, for instance, by fairly assessing coaches based on their performance while taking into account one or more features (e.g., demographic features, income level, associated social determinants, etc.) of their particular set of participants, the system and/or method can automatically and fairly determine who is performing best and compensate them commensurately.

In a fifth set of variations, additional or alternative to those described above, the system and/or method confers the benefit of learning from the best coaches, which can provide information and/or insights with which to improve the overall coaching program. This can be implemented through any or all of: coach training, the determination and/or refinement of coach performance metrics (e.g., rapport score), and/or can be implemented in any other use cases.

Additionally or alternatively, the system and method can confer any other benefit(s).

### 3. System

The system functions to enable a determination of coach quality, and can additionally function to enable a platform for coaches to coach a set of participants in any number of health programs.

A coach refers to an agent using the system to engage with and coach a set of one or more participants (equivalently referred to herein as users) in achieving one or more goals of the participant. The goals can be any or all of: selected by the participant, recommended by a coach (e.g., in accordance with self-determination theory), recommended by a third party (e.g., medical professional, employer, family member, etc.), automatically recommended and/or determined (e.g., by one or trained models), predicted, and/or otherwise determined.

The coach can refer to either or both of a human agent and a non-human agent (e.g., automated agent, robot, chat hot, etc.). For human coaches, the coach can be any or all of: a healthcare professional, a fitness trainer, a nutritionist, a therapist, a medical professional (e.g., doctor, physician, etc.), diabetes care and education specialists, clinical social workers, and/or any other individual affiliated or not affiliated with a health and/or fitness profession. In preferred variations, a participant is assigned to a human agent which remotely (and/or locally) coaches them. Additionally or alternatively, the human agent can be assisted by a non-human agent and/or a set of automated processes, which can assist or replace the human agent in performing certain tasks, such as any or all of: replying to a participant with an auto-generated message (e.g., in absence of the human agent, upon request of the human agent, in an urgent scenario, etc.), auto-filling a message initiated by a human agent, and/or performing any other suitable actions. In preferred variations, the automated content provided by non-human agents and/or automated processes is determined based on trained models (e.g., deep learning models, continuously updated deep learning models, etc.), wherein the trained models predict the most impactful response and/or proactive outreach from the human agent based on the observed impact of various strategies in advancing the user goals. Additionally or alternatively, the automated content can be determined in any other suitable way(s).

A coach supervisor refers to an agent (human and/or non-human) who monitors and/or manages a set of one or more coaches. The coach supervisor preferably monitors the coaches remotely, but can additionally or alternatively monitor any or all of them locally.

The system 100 can include and/or be configured to interface with any number of computing systems, such as any or all of: a computer, a processor, an analysis engine, a server, and/or any other suitable system(s). The computing system can be any or all of: remote (e.g., cloud computing system), local (e.g., onboard a user device, distributed among multiple user devices, etc.), or any combination of both.

The computing system preferably implements a set of one or more models, which are used in determining a coach quality and/or any or all of the intermediate metrics (e.g., OKD metrics and/or other participant outcomes, coach behavior, etc.) used in the determination of coach quality. Additionally or alternatively, the computing system can implement, store, reference, and/or otherwise utilize any suitable algorithms, equations, databases, lookup tables, or other information.

The system 100 can include a coach-participant interface, which functions to provide a platform with which a coach can interact with a set of participants. Additionally or alternatively, the coach-participant interface can include a participant dashboard, which functions to provide a platform with which a participant can interact with his or her coach. Further additionally or alternatively, the coach-participant interface can perform any or all of the following functions: providing information to a set of one or more coaches (e.g., informing them of the status of a set of users); providing information to a set of one or more participants (e.g., providing them with information associated with their health regimen(s), providing them with information from their coaches, providing them with resources, etc.); collecting information from a set of participants (e.g., aggregated information with which to update and/or train a set of models, etc.); collecting information from a set of coaches (e.g., with which to train a set of coaches); improve an efficiency of a set of coaches (e.g., through prioritization of coach tasks, through the auto-generation of a set of recommended topics, through easy-to-interpret visualizations, etc.); and/or perform any other suitable function(s).

The dashboard or dashboards of the coach-participant interface are preferably implemented on one or more user devices (e.g., associated with the coach, associated with the participant, etc.), such as through a client application executing on a user device. Additionally or alternatively, the dashboards can be otherwise implemented.

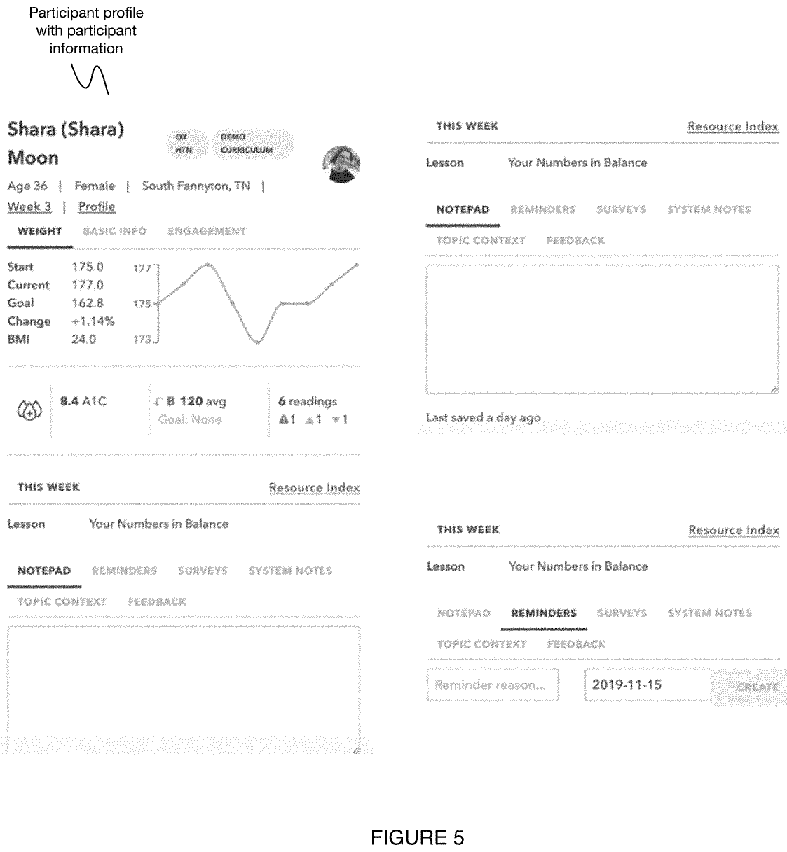

The coach-participant interface includes a coach dashboard (equivalently referred to herein as a coach interface), which functions to provide information associated with a set of one or more participants (e.g., through a participant profile as shown in FIG. 5) to the coach or coaches associated with the set of participants. The dashboard preferably additionally functions to provide a set of tools for the coach (e.g., recommended conversation topics, stored conversation topics, etc.) and to receive inputs from the coach, but can additionally or alternatively perform any other functions.

The system 100 can include and/or interface with any number of user devices, which can individually and/or collectively function to: support a dashboard, support a client application (e.g., including the dashboard), receive any or all of a set of inputs (e.g., from a set of client applications), produce any or all of a set of outputs, and/or perform any other suitable functions. Examples of the user device include a tablet, smartphone, mobile phone, laptop, watch, wearable device (e.g., glasses), or any other suitable user device. The user device can include power storage (e.g., a battery), processing systems (e.g., CPU, GPU, memory, etc.), user outputs (e.g., display, speaker, vibration mechanism, etc.), user inputs (e.g., a keyboard, touchscreen, microphone, etc.), a location system (e.g., a GPS system), sensors (e.g., optical sensors, such as light sensors and cameras, orientation sensors, such as accelerometers, gyroscopes, and altimeters, audio sensors, such as microphones, magnetometers, etc.), data communication system (e.g., a WiFi transceiver(s), Bluetooth transceiver(s), cellular transceiver(s), etc.), or any other suitable component.

The system 100 can include and/or interface with any number of sensor systems. The sensor systems can be part of a user device (e.g., accelerometer, location sensor, camera, microphone, etc.), part of a supplemental device (e.g., scale, pedometer, step counter, etc.), and/or part of any other suitable components. In some variations, for instance, one or more participant inputs are received from the sensor system. In specific examples, the sensor system includes any or all of: a connected scale for monitoring of a user's weight, a fitness tracker (e.g., step counter), a sleep tracker (e.g., as part of a smart watch), a medical device (e.g., glucose monitor, blood pressure cuff, etc.), and/or any other suitable sensor systems. Additionally or alternatively, any or all of the sensor systems and/or supplementary devices described in any or all of: U.S. application Ser. No. 15/667,218, filed 2 Aug. 2017, and U.S. application Ser. No. 17/195,156, filed 8 Mar. 2021, each of which is incorporated herein in its entirety by this reference, can be included.

### 4. Method

The method 200 functions to assess the quality of a set of coaches associated with a health coaching platform. The method 200 further preferably functions to enable a remote and/or automated assessment of coach quality, but can additionally or alternatively function to: establish a widely applicable, quantifiable metric (e.g., as described below) of participant progress; enable a comparison between coaches; enable an optimal matching between a participant and a coach; reward coaches for high quality performance; assist coaches in improving the quality of their coaching (e.g., through actionable insights into the causes of low and high coach quality); and/or perform any other suitable function(s).

The method 200 can additionally or alternatively function to perform any or all of: enable the determination and/or tracking the progress of one or more participants, enable the determination and/or tracking the progress of a coach in coaching one or more participants, and/or can perform any other suitable functions.

4.1 Method—Determining a Set of One or More Outcomes Key Drivers (OKDs) Associated with Success of a Participant S210

The method 200 can optionally include determining a set of one or more outcomes key drivers (OKDs) associated with success of a participant S210, which functions to determine a set of one or more quantifiable, comparable, and widely-applicable metric(s) with which to assess progress of a participants and thereby assess the success of the coach in bringing about participant progress. The success of the coach in bringing about participant progress reflects at least a portion of coach quality. Coach quality can additionally or alternatively take into account any other suitable factors or combination of factors, such as any or all of: a participant's dedication to and engagement with the platform; a participant's satisfaction with his or her coach (e.g., as measured in a survey); the duration of the participant's engagement with the platform; and/or any other suitable factors.

S210 is preferably performed prior to the remaining processes of the method 200, wherein the remaining processes of the method are performed based on the OKDs determined in S210. Additionally or alternatively, S210 can be performed after (e.g., in response to) any or all of the processes in the method 200, in parallel with (e.g., contemporaneously with, overlapping with, together with, etc.) any or all of the processes in the method 200, and/or otherwise performed.

S210 is further preferably performed once for all participants in the platform and optionally updated with new information and/or platform expansions (e.g., into other health conditions). Additionally or alternatively, S210 can be performed for any or all of: each participant, each participant goal (e.g., weight loss, diabetes management, etc.) and/or other information, each instance of the method 200, multiple times in the method 200, and/or at any other suitable times. Further additionally or alternatively, the method 200 can be performed in absence of S210.

The set of OKDs is a set of one or more metrics and/or parameters, which are found and/or designed to correlate with one or more desired outcomes (e.g., participant goals) of one or more participants in the coach platform. Additionally or alternatively, the set of OKDs can be found and/or designed to correlate with desired outcomes of the platform, such as any or all of: participant engagement, profits generated by the coaching platform, coach efficiency, and/or any other factors.

Each OKD is preferably associated with (e.g., correlated with) one or more of: the health of a participant, the fitness of a participant, the medical state of a participant (e.g., physical state, mental state, etc.). Additionally or alternatively, one or more OKDs can be associated with any other suitable factors. Each OKD is preferably widely applicable to a set of multiple participants (e.g., all participants associated with a single coach, all participants in the platform, all participants associated with a particular goal or similar goals, etc.), if not all participants, such that a coach quality metric can be determined which is comparable among multiple coaches. Additionally, this can function to enable a progress metrics of a participant to be comparable among multiple participants. Additionally or alternatively, OKDs can be assigned to and/or otherwise associated with participants based on any or all of: an offering of the health platform (e.g., weight loss, dietary restriction guidance, diabetes management, blood pressure management, cardiac condition management/prevention, management and/or prevention of other health conditions, etc.); an experience and/or background of one or more coaches (e.g., clinical training in obesity, clinical training in diabetes, etc.); and/or any other suitable information.

OKDs can be associated with any or all of: a single participant (e.g., wherein each coach is associated with one or more OKDs for each participant), multiple participants such as all participants assigned to a coach (e.g., wherein each coach is associated with one of each type of OKDs applying to his or her whole cohort of participants), and/or OKDs can be otherwise determined and/or associated.

The OKDs are preferably configured to distinguish between factors outside of the coach's control and factors within the coach's control, such that any or all of the metrics produced by the OKDs are reflective only of (or mostly/majorly reflective of) the factors within the coach's control, such that the coach's contribution in helping the participant achieve an outcome can be fairly and comparably (e.g., among other coaches, for a coach at different times or with different participant cohorts, etc.) determined.

As such, determining each OKD preferably includes adjusting for (e.g., normalizing for, filtering for, etc.) a set of features and/or parameters (equivalently referred to herein as metrics) which may significantly vary among a group of participants. These identified features are further preferably unchangeable and/or not influenced by (e.g., not able to be improved upon) with coaching and/or the performance of a particular coach. Identifying and accounting for these features functions to enable a proper (e.g., uniform, fair, etc.) assessment of the performance of coaches and therefore enable the determination of a reliable, reproducible, and comparable (e.g., among different coaches, among different participants associated with a coach, for a coach at different times and/or with different cohorts of participants, etc.) coach quality metric for each coach. For instance, features of the user (e.g., wealth, high metabolism, etc.) which would bias them toward relatively high outcomes (e.g., high weight loss, good health, etc.) independent of the coaching they receive can be taken into account such that the actual outcomes of the participant (e.g., actual weight loss) are decreased (e.g., scaled down, multiplied by a factor between 0 and 1, etc.) when evaluating the quality of the coach, whereas features of the user (e.g., poverty, low metabolism, etc.) which would bias them toward relatively low outcomes (e.g., low amount of weight loss) can be taken into account such that the actual outcomes of the participant (e.g., actual weight loss) are increased (e.g., scaled up, multiplied by a factor above 1, etc.) when evaluating the quality of the coach. This helps fairly assess the contributions of the coach.

The set of features are preferably determined based on the particular participant goal and/or outcome (e.g., weight loss, weight gain, healthy eating, high blood pressure management, low blood pressure management, etc.) being examined, but can additionally or alternatively be determined based on any other suitable factors.

The set of features and/or parameter can include demographic information associated with the participants, such as any or all of: age, sex, and race. Additionally or alternatively, the set of features and/or parameters can include lifestyle information (e.g., occupation, activity/fitness level, schedule, etc.), other inputs, body composition (e.g., current weight, height, measurements, body mass index [BMI], ectomorph vs. endomorph vs. mesomorph, etc.) or other body parameters, medical or biometric information (e.g., blood pressure, glucose level, presence of a medical condition, etc.), income level, socioeconomic conditions associated with the user, social determinants of health associated with the user (e.g., local availability of health food options, local availability of fitness opportunities such as gyms and/or outdoor spaces, income level, education level, marital status, parental status, residential conditions, etc.), geographic/location information of user (e.g., location of residence, zip code of residence, proximity to a grocery store with healthy food options, proximity to gym, etc.), employment information (e.g., employed vs. not, hours spent working vs. amount of free time, number of jobs, etc.), any existing health conditions (e.g., cancer, high blood pressure, low blood pressure, Type-I diabetes, Type-II diabetes, etc.), and/or any other suitable information.

In some examples, for instance, location information (e.g., zip code of residence) of the user is used to determine a quality of the social determinants in that location, wherein the quality of the social determinants is used by one or more models in the determination of OKDs (e.g., as described below).

In a first variation, for instance, one goal of the health program as a whole and/or for a particular subset of participants (e.g., overweight participants, diabetic participants, obese participants, etc.) might be to maximize a weight loss for each participant through coaching. There are various factors which might help or hinder the participant from losing weight, which are not within the control of the coach. One example of an OKD that can take these factors into account is through the determination of an adjusted weight loss which eliminates these variable factors from consideration in the patient's weight loss, such that an objective quality of the coach in achieving this goal can be determined. In specific examples, for instance, any or all of the following features could be taken into account when determining an adjusted weight loss for participants: a sex of the participant (e.g., such that an adjusted weight loss is increased/boosted for females relative to their actual weight loss since it has been shown that females lose less weight overall and/or lose weight slower than men); an age of the participant (e.g., such that an adjusted weight loss is increased/boosted for elderly participants who are not able to perform rigorous exercise); a race and/or cultural characteristics of the participant (e.g., based on studies showing which races have historically higher metabolisms, based on cultural diets, etc.); body composition information (e.g., such that an adjusted weight loss is decreased for participants with a large starting weight as they are able to lose a larger amount of weight quickly, etc.); metabolism information (e.g., such that an adjusted weight loss is boosted/increased for those struggling with a metabolic condition such as a thyroid condition); financial information such as income level (e.g., such that an adjusted weight loss is boosted/increased for those who cannot afford expensive health foods and/or gym equipment/memberships, such that an adjusted weight loss is decreased for those with a low-calorie private chef, etc.); employment information and/or workload (e.g., to determine an opportunity for the participant to exercise, etc.); existing health conditions (e.g., cancer); and/or any other suitable information.

In a preferred set of variations, the set of OKDs is associated with a set of goals associated with participants in the health platform. These goals can be any or all of: predetermined (e.g., based on a particular program in which the participant is enrolled in the digital health platform, based on a subscription level and/or type of the participant, etc.); dynamically determined (e.g., based on interests of the participant, based on progress of the participant, based on the coach, etc.); any combination; and/or otherwise determined.

The progress of each participant is preferably measured through at least one OKD, but can additionally be measured through multiple OKDs, in absence of an OKD, and/or through any other suitable metrics. The OKDs assigned to a participant can be any or all of: set (e.g., predetermined), dynamic (e.g., based on participant progress), and/or any combination of both. The OKD(s) associated with a participant can be the same among any or all participants, different among any or all participants, have some overlap among participants, be similar among participants, be determined individually for each participant, be the same among all participants in the platform, be the same among all participants in a particular program of the platform, be the same among all participants assigned to a particular coach, randomly assigned, and/or be otherwise associated with participants.

The set of OKDs preferably includes one or more health and/or fitness OKDs, such as those associated with any or all of: weight loss, BMI loss, healthy eating, fitness and/or physical activity, muscle toning, and/or any other parameters reflecting the participant's progress in achieving a healthier lifestyle. Specific examples of these OKDs can include, but are not limited to, for instance, any or all of: an adjusted weight loss, an adjusted BMI loss, an adjusted muscle gain, an adjusted improvement in healthy eating (e.g., based on a participant's meal planning, based on a calorie intake, etc.), an adjusted fitness level, and/or any other suitable parameters.

In specific examples, for instance, the progress of one or more participants is assessed through a predetermined set of OKDs associated with a program of the health platform, such as through an adjusted weight loss metric for participants in a weight loss program). Additionally or alternatively, participants can be otherwise associated with any suitable OKDs.

The set of OKDs can additionally or alternatively include one or more OKDs associated with a medical condition of a participant (e.g., pathology-specific OKD, disease-specific OKD, OKD associated with a biometric parameter such as glucose level, etc.). Examples of this can include, but are not limited to, for instance, any or all of: an adjusted A1C level, an adjusted systolic blood pressure, an adjusted diastolic blood pressure, an adjusted pain and/or pain reduction (e.g., chronic pain, temporary pain, pain associated with a particular condition and/or procedure, etc.), an adjusted PHQ-9 score, an adjusted weight gain (e.g., for participants with disordered eating), and/or any other suitable parameters.

In a first set of variations, the set of OKDs includes a normalized weight loss parameter, herein referred to as adjusted weight loss. The adjusted weight loss functions to capture the true impact of the coach in helping a participant or set of participants within the participant's particular circumstances achieve a health goal (e.g., losing weight). This can serve as a significant improvement to conventional systems and methods which, for instance, merely track a percentage of weight lost by each participant and aggregate these percentages to determine the success/quality of a coach, without taking into account any of the factors which may naturally increase the ease with which a participant may lose weight (which may inflate a determined coach quality metric), or conversely the factors which may naturally increase the difficulty of a participant losing weight (which may result in a low coach quality metric). In preferred embodiments of this variation, the adjusted weight loss is normalized with respect to demographic information associated with the participant which has been shown (e.g., in literature, in research, historically demonstrated for the particular participant, based on aggregated participant information, etc.) and/or predicted to have an effect on a participant's ability/propensity (e.g., natural ability based on demographics, baseline ability based on current weight/fitness level, etc.) to lose weight (e.g., in light of a medical condition of the participant, in light of the patient's age, in light of the patient's lifestyle, in light of the patient's sex, in light of the patient's race, etc.). Additionally or alternatively, any other suitable parameter associated with weight loss and/or body composition change (e.g., muscle gain, fat loss, etc.) can form an OKD (e.g., through a set of normalization processes, based on normalization factors described above, based on different normalization factors as described above, etc.), such as an any or all of: an adjusted body fat percentage loss, an adjusted body muscle percentage gain, an adjusted decrease in one or more body measurements, and/or any other suitable parameters.

In specific examples, for instance, an adjusted weight loss is determined accounts for a number of factors which may result in the determination of a “too low” coach quality metric, such as any or all of: participant sex (e.g., female), muscle composition, historical information (e.g., previous weight loss journey(s)), health (e.g., unhealthy, experiencing a hormone imbalance, experiencing a thyroid condition, diagnosed with polycystic ovarian syndrome [PCOS]), physical ability (e.g., disabled), and/or any other factors. The adjusted weight loss can further account for any number of factors which may result in the determination of a “too high” coach quality metric, such as any or all of: participant sex (e.g., male), muscle composition, historical information (e.g., previous weight loss journey(s)), health (e.g., healthy), and/or any other factors.

In a second set of variations, additional or alternative to the first set, a medical parameter is determined for any or all participants (e.g., all participants associated with a particular medical condition, all participants collecting data associated with a particular medical condition, etc.), which can function to quantify a participant's progress with managing a medical condition and thereby appropriately assess the coach's role in achieving this. Additionally, the medical parameter (and/or any other parameters/metrics) can optionally be used as a configurable optimization function in determining automated content (e.g., as described above). The medical parameter can be adjusted (e.g., normalized), such as based on any or all of: factors described previously (e.g., demographic information, historical information, etc.), comorbidities, medications, historical health and/or medical information, and/or any other suitable factors. Additionally or alternatively, the medical parameters can be non-adjusted direct parameters (e.g., as received directly from a sensor system). In examples, the medical parameter can include any or all of: a glucose level (e.g., adjusted A1C level), a blood pressure level (e.g., blood pressure, adjusted blood pressure, etc.), an adjusted heart rate parameter (e.g., heart rate, adjusted heart rate, etc.), and/or any other suitable parameters.

In a set of variations, a medical parameter of an adjusted A1C level is determined for at least a subset of participants associated with diabetes management.

4.2 Method—Determining a Set of Models Associated with the Set of OKDs S220

The method 200 can optionally include determining a set of models associated with the set of OKDs S220, wherein the models function to determine the relationship (e.g., algorithm, equation, mapping, etc.) between the inputs involved in determining an OKD to the value of the OKD. Additionally or alternatively, determining the set of models can function to: determine which metrics best serve as an OKD, determine proper correlations (and/or absence of correlations) between factors which differentiate participants (e.g., demographic information) from each other and the parameter(s) being used to determine coach quality, and/or perform any other function.

S220 is preferably performed in response to and based on the OKDs selected in S210, further preferably prior to any or all of the remaining processes of the method, such that the method 200 can be performed with the model(s) determined in S200. Additionally or alternatively, S220 can be performed at any or all of: independently and/or in absence of S210, in parallel with S210, after any or all of the processes described below, multiple times during the method 200, and/or at any other times. Further additionally or alternatively, the method 200 can be performed in absence of S220.

The set of models preferably includes one or more trained models, such as any or all of: deep learning models, machine learning models (e.g., regression models, reinforcement learning models, etc.), neural networks, and/or any other suitable models. Additionally or alternatively, the set of models can include and/or produce any or all of: rule-based models (e.g., programmed models), probabilistic models, statistical models, algorithms, equations, decision trees, and/or any other tools for determining a value of an OKD. The models are preferably implemented at a computing system (e.g., a remote computing system), but can additionally or alternatively be implemented at any or all of: multiple computing systems, one or more processing systems (e.g., processing system of a mobile device executing a client application, among multiple processing systems, etc.), and/or any in any other suitable way with any suitable components.

The models are preferably determined (e.g., trained) based on historical information associated with a set of aggregated participants, wherein the historical information can include any or all of: demographic information associated with the participant; sensor information associated with the participant (e.g., weight measurements, glucose measurements, etc.), which can be collected at a set of supplementary sensors (e.g., scale, biometric sensors, glucose monitor, etc.), for instance; information associated with participant participation in the coaching platform (e.g., participant progress, participant goals, participant success in achieving goals, etc.); long-term information associated with participant (e.g., duration of time weight loss was maintained, participant health later in time, etc.); any of the inputs described previously; and/or any other suitable information. With this aggregated information, the models can then be configured to determine what outcomes (e.g., weight loss, other goal progress, etc.) are expected for each user based on features (e.g., demographic information, race, sex, socioeconomic condition, etc.) of that user, particularly features which are outside of the coach's control and/or his or her responsibilities in coaching the participant.

Each OKD is preferably associated with its own model, wherein the model can be adjusted for and/or adapted to any or all of the coaches. In specific examples, for instance, an adjusted weight loss is determined with an adjusted weight loss model, an adjusted A1c level is determined with an adjusted A1c model, and so forth. Additionally or alternatively, a single model (e.g., omnibus model) can be used to determine multiple OKDs, each coach can have his or her own model(s) (e.g., for all OKDs, for each OKD, etc.), each participant can be associated with his or her own model (e.g., for each OKD, for all OKDs, etc.), and/or the models can be otherwise configured.

Any or all of the models (e.g., trained models, untrained models, etc.) preferably implement predictive modeling, wherein the predictive modeling functions to predict a value of one or OKDs based on participant information, wherein a coach quality can ultimately be determined (e.g., partially determined, fully determined, etc.) based on a comparison between the actual value of the parameter (e.g., currently, historically, at a future time point) and the predicted value. In variations, for instance, an adjusted weight loss (expected weight loss) can be predicted for each participant based on participant information and a set of predictive models trained on numerous participants associated with a diversity of parameters (e.g., sex, starting weight, age, race, region, social factors, proximity to a food desert, etc.), wherein the actual weight loss of the participant throughout the program can be compared with the adjusted weight loss to determine if the participant has met the expected weight loss, exceeded the expected weight loss (e.g., high coach quality), underperformed relative to the expected weight loss (e.g., low coach quality), and/or otherwise performed relative to the expected weight loss. Additionally or alternatively, any other OKDs or other parameters can be predicted and used for comparison. In variations including a medical parameter OKD (e.g., an adjusted A1C value), for instance, a predicted value of the medical parameter can be determined with a set of one or more models, wherein a comparison between the predicted value and an actual value can be used to assess the quality of the coach working with the participant during that time.

Additionally or alternatively, determining any or all of the adjusted parameters can be performed with the known outcome. For instance, determining an adjusted weight loss can include receiving the actual weight loss at the model, and adjusting its value (e.g., normalizing the value) based on features of the participant. The coach quality can then increase as the adjusted weight loss for his or her participants increases.

Additionally or alternatively, the model(s) can otherwise suitably determine the OKDs.

Determining and/or refining (e.g., training) the set of models can optionally include developing a set of multiple different models, testing the models with a diverse set of participant populations, and determining a predictive model which produces results consistent with the scenarios from the trained data.

Additionally or alternatively, the set of models can be otherwise determined.

The set of models preferably includes one or more statistical models, wherein the statistical models are able to be updated as new information is received (e.g., from the same participants, from new participants, from a new coach, from the same coach, etc.). In some variations, for instance, the model includes a Bayesian model, wherein the Bayesian model provides tools with which to update beliefs of the model in the evidence of new data, as well as takes into account conditional probabilities in determining the OKDs. Additionally or alternatively, the model can include other statistical models and/or probabilistic models, other conditional probability models, frequentist models, and/or any other suitable models.

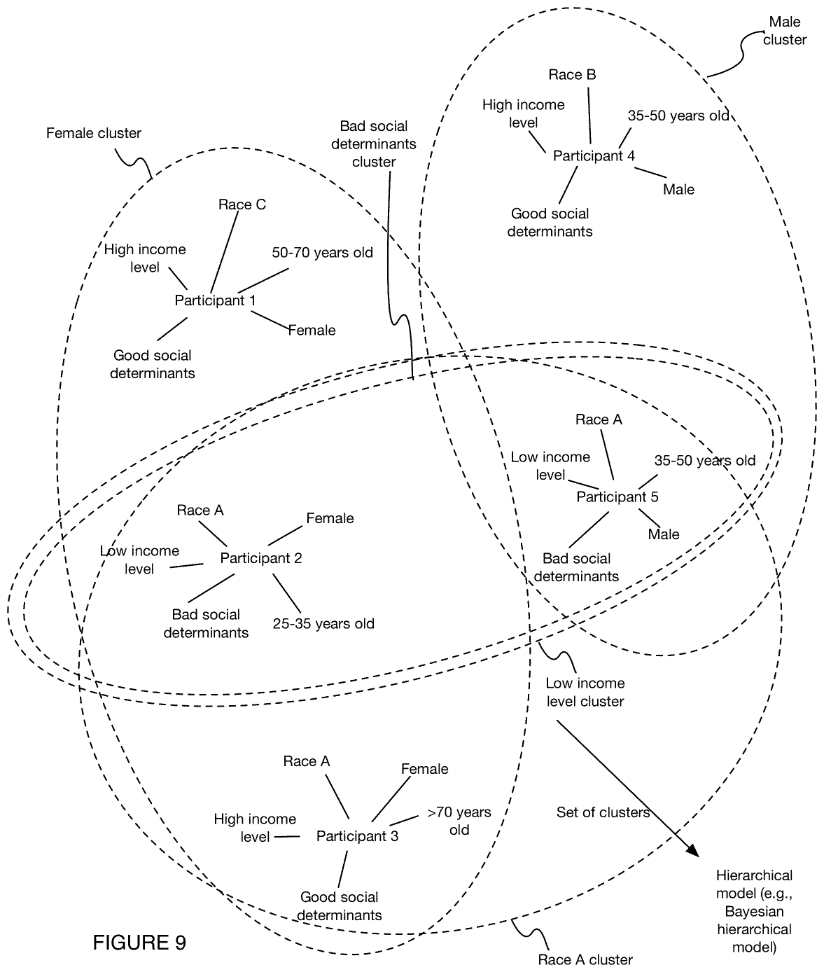

The set of models further preferably includes one or more hierarchical models, wherein a hierarchical model functions to enable data (e.g., from multiple participants) to be organized (e.g., grouped) into clusters (e.g., as shown in FIG. 9). This can subsequently enable the influence of the clusters on the data points contained in them to be taken into account in the analysis (e.g., in a statistical analysis). This can confer benefits in determining OKDs for coaches who are assigned multiple participants, for instance, as the model can cluster the information from the participants based on shared features and/or attributes and/or similarities (e.g., age, sex, social determinants, etc.), such as any or all of those described above. Alternatively, general linear models (in which observations are considered independently of each other) and/or any other models can be used. The hierarchical model can optionally have cross-level interactions, any number of stages (e.g., two-stage, three-stage, greater than three-stage, etc.), and/or be otherwise configured. The hierarchical model can be a regression hierarchical model, a non-regression hierarchical model, and/or any other suitable hierarchical model.

Additionally or alternatively, the model can include any other suitable models.

In a preferred set of variations, the set of models includes one or more Bayesian hierarchical models. In specific examples, each OKD is associated with its own Bayesian hierarchical model. Alternatively, a Bayesian hierarchical model can be associated with multiple OKDs.

In a first set of variations of S220, a set of one or more models are tested and/or trained (e.g., with participant weight loss data) to determine an optimal model for predicting a weight loss of each participant based on the information associated with the participant (e.g., demographic information, social determinants, body parameters, etc.), referred to as an adjusted weight loss metric. The optimal model and/or algorithms resulting from the optimal model can be used subsequently in the method when determining an adjusted weight loss metric for each of the set of participants.

Additionally or alternatively, in a second set of variations, S220 includes training a set of one or more machine learning models based on historical aggregated information from a set of participants. The historical aggregated information is preferably associated with multiple coaches, but can alternatively be associated with a single coach.

Additionally or alternatively, in a third set of variations, S220 includes determining a set of programmed models based on historical aggregated information from a set of participants. The historical aggregated information is preferably associated with multiple coaches, but can alternatively be associated with a single coach.

4.3 Method—for Each of a Set of Coaches, Determining a Baseline Quality Associated with the Coach S230

The method 200 can optionally include, for each of a set of coaches, determining a baseline quality associated with the coach S230, which can function to enable a proper determination of a coach quality and/or a progression of coach quality. Additionally or alternatively, the baseline quality can be used in implementing and/or adapting one or more models (e.g., as described above), selecting one or more models, and/or can be otherwise suitably used.

S230 is preferably performed prior to S270 such that the baseline quality can be compared with the coach quality metric determined in S260. Additionally or alternatively, S230 can be performed prior to S260, prior to S250, prior to S240, in response to S230, prior to S220 and/or during S220 (e.g., to determine any or all of the models), prior to S210, multiple times in the method 200, and/or at any other suitable times. Alternatively, the method 200 can be performed in absence of S230.

The baseline coach quality can be determined based on any or all of: a coach's background (e.g., career/professional background, career in a health field, career in a fitness field, career in a medical field, years of experience in a particular field, etc.); a coach's experience level (e.g., number of years coaching, number of participants coached, etc.); a progress of previous participants of the coach; and/or any other suitable parameters associated with coach.

Additionally or alternatively, the baseline coach quality can include a coach quality metric determined previously with a prior iteration of the method 200 and/or any other method.

Determining a baseline quality can optionally include making one or more assumptions and/or comparisons between the coach and other coaches, such as based on a background and/or experience of the coach. In some variations, for instance, in determining a baseline quality for a new coach, the quality associated with one or more coaches having the same or a similar background (e.g., prior job experience, education, etc.) is used as a baseline quality. This comparison can additionally or alternatively be used to determine one or parameters in the one or models used to assess the OKDs and/or coach quality as described below. In specific examples, for instance, a comparison with other coaches is used to determine a set of priors for a Bayesian hierarchical model associated with a new coach (e.g., as triggered based on a coach experience with the platform falling below a predetermined threshold).

In some variations, a baseline coach quality metric is determined for each coach, wherein assessing coach quality later in the method 200 includes comparing a current coach quality with the baseline coach quality (e.g., to determine the coach's progress).

Additionally or alternatively, the baseline coach quality metric can be otherwise suitably used.

### 4.4 Method—Collecting a Set of Inputs S240

The method 200 can include collecting a set of inputs S240, which functions to collect information with which to assess participant progress and therefore enable a determination of coach quality.

S240 is preferably performed continuously during the method 200, such as whenever inputs are received from a participant. Additionally or alternatively, S240 can be performed in response to one or more check-ins (e.g., scheduled weigh-ins, scheduled appointments, scheduled measurement collection from a biometric device, etc.), milestones of a participant (e.g., completion of a portion of the coaching program), triggers (e.g., from a coach, from a participant, from the system, from a coach's manager, etc.), at random intervals, and/or at any other suitable times. Additionally or alternatively, S240 can be performed in response to any other processes of the method, prior to any other processes of the method, once during the method, and/or at any other suitable times. Further additionally or alternatively, the method 200 can be performed in absence of S240.

The set of inputs can be received from any or all of: a set of one or more participants, a set of one or more coaches, a computing system (e.g., as generated by the set of models), external information sources (e.g., census information, online information, etc.), a user device and/or client applications (e.g., a meal tracker client application, a fitness tracker client application, sleep tracker client application, etc.), supplementary devices (e.g., smart watch, blood pressure monitor, glucose meter, insulin pump sensor, scale for measuring user weight, etc.), databases (e.g., lookup tables, medical files, etc.), and/or any other suitable information sources.

The set of inputs can be received at any or all of: one or more user devices, a computing system (e.g., a remote computing system, computing system of a user device, etc.) and/or server (e.g., remote server, cloud-based server, etc.), a dashboard (e.g., coach dashboard, participant dashboard, etc.), and/or any other suitable locations.

Any or all of the set of inputs can be collected continuously (e.g., at a predetermined frequency, upon being input into a dashboard by a coach and/or a participant, at an intermittent frequency, etc.). Additionally or alternatively, one or more inputs can be collected once (e.g., participant demographic information), in response to a trigger, upon being updated, and/or at any other suitable times.

The set of inputs can include any number of coach inputs, which are received from and/or otherwise associated with one or more coaches (e.g., automatically generated and/or determined) and can function to guide the participants in their regimens and achieving their health goals or other goals. The coach inputs can optionally be used in the method 200 to determine the quality of coach interactions with his or her participants. Coach inputs can include any or all of: articles to send to a participant (e.g., to help them with a particular topic, to help them lose weight, to guide them in performing a fitness routine, etc.), surveys to send to a participant, messages to send to a participant, reminders to send to a participant, and/or any other suitable inputs. Additionally or alternatively, the coach inputs can include notes and/or other documentation configured to help a coach remember and/or track information, such as the coach's thoughts about a participant (e.g., his or her progress, topics to check in on, etc.). Further additionally or alternatively, the set of coach inputs can include coach preferences (e.g., dashboard format preferences) and/or coach information (e.g., demographic information, historical information, prior messages, etc.).

The coach inputs can additionally or alternatively include information associated with the coach, which can be used, for instance, in tailoring any or all of the models to the coach (e.g., to set priors in a Bayesian hierarchical model, to train a model, etc.), such as any or all of: coach demographic information, coach experience in the platform and/or elsewhere such as in a career (e.g., years of experience, number of patients coached, tenure, level, etc.), and/or any other information.

The coach inputs can further additionally or alternatively include information associated with the coach's activity in the platform, such as messaging information between the coach and his or her participants (and/or any other communications between the coach and his or her participants). This can include, for instance, any or all of: time spent logged in to the coach program, number of messages exchanged with one or more participants, length of messages exchanged with one or more participants, time between reaching out to a participant, frequency of interactions with a participant, the tone (e.g., encouraging, demeaning, active listening, etc.) of the coach's messages to one or more participants, the types of resources sent to the participants and whether or not they were relevant and/or useful, and/or any other suitable information.

Additionally or alternatively, the coach inputs can include any other suitable information.

The coach inputs can be any or all of: determined (e.g., selected, developed, etc.) by the coach, determined by a computing system (e.g., through a set of models), and/or any combination of both (e.g., suggested to a coach and selected by the coach).

The set of inputs can include any number of participant inputs, which are received from participants and can function to provide information to a coach, provide information for a set of models to analyze, provide information to train a set of models, provide information to be aggregated with information from other users, and/or perform any other suitable function.

The participant inputs further preferably function to determine a set of features associated with each of the set of participants, such as any or all of the features described above, wherein the features can be used to determine OKD values which reflect the coach's contributions in outcomes associated with the participant (and eliminate contributions—negative or positive—from features outside of the coach's control). In specific examples, for instance, these features can be used to organize the coach's participants into a set of clusters in a Bayesian hierarchical model for determining an OKD.

As such, the participant inputs can include any or all of the feature information described above, such as, but not limited to, any or all of: demographic information, bodily composition information, socioeconomic information, existing conditions and/or comorbidities, education level, and/or any other suitable information.

The participant inputs can be any or all of: provided by the participant, provided by an external source (e.g., healthcare facility visited by the participant, sampled at a sensor system of a user device and/or external device, etc.), determined by a computing system (e.g., through a set of models), and/or any combination of both (e.g., provided by a participant and processed at a computing system).

The participant inputs can optionally include one or more clinical outcomes (e.g., diabetes, depression risk, medical records, etc.). In some variations, clinical outcomes are received in response to a user providing consent for the system to access clinical records (e.g., from a healthcare facility, from a medical records database, etc.). Additionally or alternatively, the clinical outcomes can be otherwise received (e.g., directly from the participant, etc.).

The participant inputs can optionally include participant preferences, such as goals and motivations (e.g., look better, lose weight, complete a particular fitness activity, increase strength, increase endurance, reduce severity of a medical condition, etc.). The participant preferences can be determined based on any or all of: clickstream data (e.g., within a client application containing the participant dashboard, within external client applications, on the internet, etc.), survey information, and/or any other suitable sources.

The participant inputs can optionally include social determinants, which can function to tailor the coaching provided to the participant. These can include any or all of: regional parameters associated with the participant's location (e.g., food desert, food oasis, proximity to a healthy food source), lifestyle parameters (e.g., commute time, occupation, etc.), income level, geographical information, and/or any other suitable parameters. The social determinants can be collected from the participant, from 3^{rd} party and/or public data sources (e.g., census information, neighborhood information, employment information, LinkedIn, internet searches, etc.), and/or from any other information sources. In some variations, for instance, any or all of these social determinants can significantly affect a participant's ability or predicted ability to make positive change in leading a health lifestyle (e.g., thereby contributing to an adjusted weight loss metric). If the participant lives in a food desert, for instance, the coach may provide resources specific to this type of region, such as recipes which do not include ingredients difficult for the participant to easily access.

The participant inputs can optionally include time-varying behaviors (e.g., habits), such as temporal seasonality (e.g., weekly seasonality where participant is offline for a week with no access to scale for weigh-ins due to an occupation such as a flight attendant, truck driver, etc.; monthly seasonality; etc.). The time-varying behaviors can be determined based on direct inputs from the participant, information collected at client applications (e.g., calendar), employment information of the participant, sensor information associated with the participant (e.g., elevation, GPS information, pedometer information, etc.), and/or any other suitable information.

The participant inputs can include lifestyle data, such as any or all of: food consumed by the participant (e.g., tracked meals, tracked calories, photos of food, etc.), exercise performed by the participant (e.g., as input by the participant, as detected through one or more sensors such as a pedometer, etc.), and/or any other suitable information.

The participant inputs preferably include outcome information which reflects his or her progress in the program. The type of outcome information can be dependent on any or all of: the particular program in which the participant is involved, any conditions and/or criteria which apply to the participant, the goals of the participant, input from a coach, and/or any other suitable information. Additionally or alternatively, the outcome information can be the same for all participants, for all participants of a particular coach, and/or otherwise determined.

The outcome information can be any or all of: automatically collected from one or more devices and/or supplementary devices (e.g., weight scale, biometric sensors, medical devices, etc.), manually entered by a participant, and/or otherwise received or determined.

The outcome information is preferably a measurement or other input which corresponds to a particular OKD associated with the participant. For a participant for which an adjusted weight loss is to be determined, for instance, the outcome information can be his or her actual weight and/or weight loss, such as that determined by a scale. For a participant for which an adjusted A1C value is to be determined, the outcome information can be his or her actual A1C level. Additionally or alternatively, the outcome information can include any other parameters.

In a first set of variations, a set of inputs is collected for each coach, wherein the set of inputs for each coach includes information from each of the participants (e.g., demographic information, sensor information, etc.) that the coach is coaching and information associated with the coach (e.g., messages sent to participants). In a set of specific examples, the set of inputs collected for each coach includes at least demographic information (e.g., age, race, sex, etc.) associated with each participant, sensor data associated with each participant (e.g., weight of the patient, biometric parameter of the participant, etc.), and coach-participant engagement information associated with each participant (e.g., number of messages exchanged between the coach and the participant, content of message exchanged between the coach and the participant, etc.). Additionally, the set of inputs can include survey results from one or more participants, coach activity information (e.g., number of hours logged per day on the coach platform, average response time, etc.), and/or any other suitable information.

In a second set of variations, additional or alternative to the first, the set of inputs includes at least participant outcome information associated with the OKDs relevant to the participant, and feature information associated with the participant which may have influenced (e.g., positively, negatively, etc.) the outcome information. Additionally, any other information (e.g., coach inputs, coach-participant messaging information, etc.) can be received.

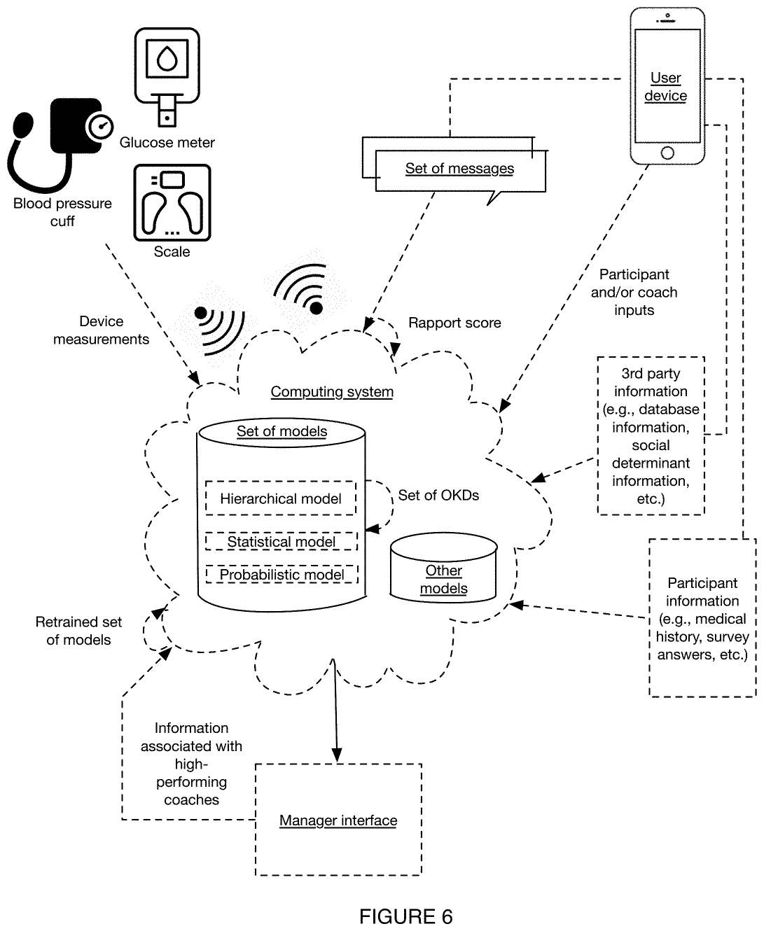

In a third set of variations, a variation of a set of inputs received is shown in FIG. 6.

Additionally or alternatively, any other inputs can be received in S240.

### 4.5 Method—Determining a Set of Metrics Based on the Information S250

The method 200 includes determining a set of metrics based on the information S250, which can ultimately functions to enable a determination of coach quality, but can additionally function to enable a determination of any or all of: a participant performance/progress metric; a coach engagement metric with one or more participants; a coach performance metric; and/or any other suitable metrics.

S250 is preferably performed in response to and based on S240, but can additionally or alternatively be performed in response to any other suitable processes, prior to any suitable processes, contemporaneously with (e.g., partially overlapping with, fully overlapping with, etc.) any other suitable processes, and/or performed at any other suitable times. Additionally or alternatively, S240 can be performed at any or all of: continuously, at a predetermined frequency (e.g., weekly, daily, monthly, etc.), at a set of predetermined intervals, at a set of random intervals, in response to a trigger (e.g., a weigh-in of a participant, prompting of a coach manager, at the time of a performance review of a coach, etc.), and/or at any other suitable times.

The set of metrics are preferably determined (e.g., calculated) based on one or more of the inputs described in S240, but can additionally or alternatively be determined based on any other suitable information. One or more of the metrics are preferably determined based on a set of models as described above, but can additionally or alternatively be determined with any other suitable models, algorithms, equations, lookup tables, databases, decision trees, and/or in any other suitable way.

The set of metrics can include one or more short-term metrics, which refer to metrics that can be calculated within a short time frame (e.g., immediately, frequently, continuously, etc.). Examples of this include time spent by coach in a coach dashboard, an average response time of a coach, and the time between a coach receiving and responding to a participant message. The set of metrics can additionally or alternatively include one or more long-term metrics, which refer to metrics that are calculated based on inputs collected over a long time frame (e.g., over weeks, months, etc.). Examples of long-term metrics can include one or more OKDs, survey results, and/or any other metrics. Additionally or alternatively, any of the long-term metrics can be determined based on inputs collected over short time frame, any of the short-term metrics can be determined based on inputs collected over a long time frame, and/or any other metrics can be determine.

In some variations, a set of short-term metrics and a set of long-term metrics are collected, wherein the short-term metrics are analyzed in a binary fashion (e.g., checked to ensure coach is reaching a minimum engagement threshold), whereas the long-term metrics are further processed and/or analyzed to determine a coach quality. Additionally or alternatively, any or all of the short-term metrics can be further processed and/or analyzed, any or all of the long-term metrics can be analyzed in a binary fashion, and/or the metrics can be analyzed in any other suitable way(s).

The set of metrics can include any number of coach metrics. One or more coach metrics can be determined based on coach dashboard activity, such as any or all of: a coach's log in/log out activity of a coach dashboard (e.g., via a client application); one or more temporal parameters (e.g., amount of time spent logged in to coach dashboard, amount of time coach is actively engaging with participants [e.g., time spent writing a response, time spent reading a participant message, etc.], time between 2 consecutive messages, etc.); and/or any other suitable dashboard parameters.

Additionally or alternatively, one or more coach metrics can be determined based on participant information, such as one or more tags or labels associated with the participant. If a patient is labeled as a critical, for instance, a fast response time of a coach might be extra valuable and therefore factored into the calculation of the coach metric and/or the coach quality.

In addition or alternative to the coach metrics, the set of metrics can include one or more participant metrics, which can function to reflect any or all of: the current state of the participant within the coaching platform, a progress of the participant while taking part in the coaching platform, an engagement of the participant with the coaching platform, and/or any other suitable information.

The participant metrics preferably include a set of one or more OKDs, further preferably one or more of the OKDs described above, but additionally or alternatively any other suitable OKDs. In specific examples, for instance, S250 includes determining one or more adjusted parameters with a set of models determined in S220.

The set of OKDs can be determined for each participant, for all participants in a coach cohort, and/or for any other groupings of participants. In some variations, for instance, an OKD metric (e.g., adjusted weight loss) is determined for all of the participants assigned to a coach (e.g., based on aggregating [e.g., averaging, summing, etc.] individual OKD metric values from participants). In specific examples, for instance, an adjusted weight loss is determined for each coach based on his or her cohort. Additionally or alternatively, any other OKD metrics (e.g., adjusted A1C value) can be determined for each coach. Further additionally or alternatively, an OKD metric can be determined for each participant (e.g., and aggregated to determine a single OKD metric of each type for each coach) and/or otherwise determined.

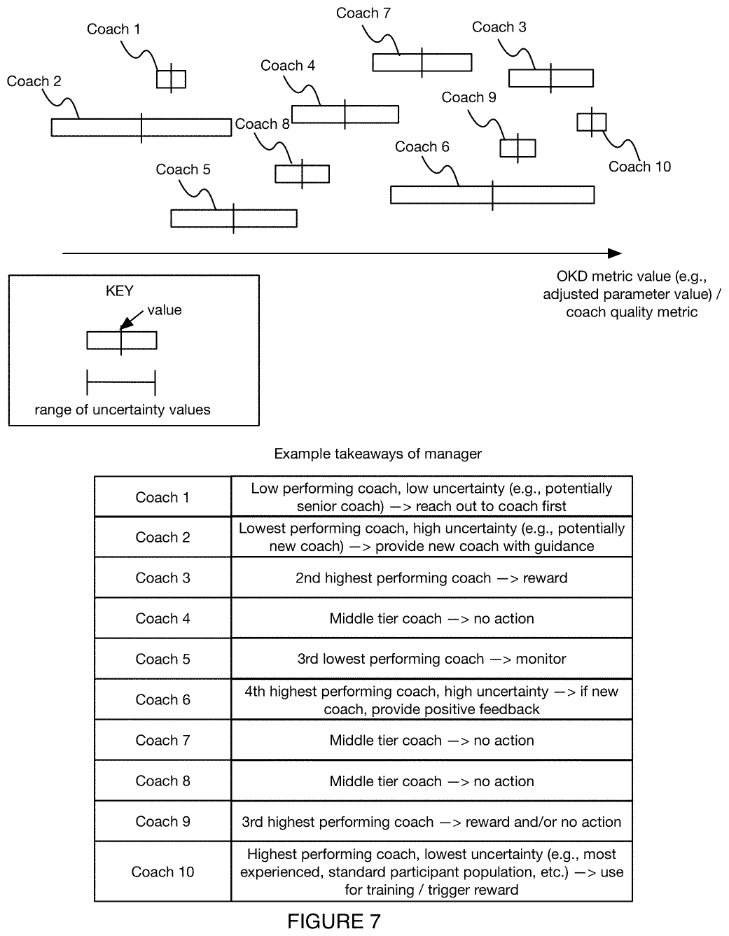

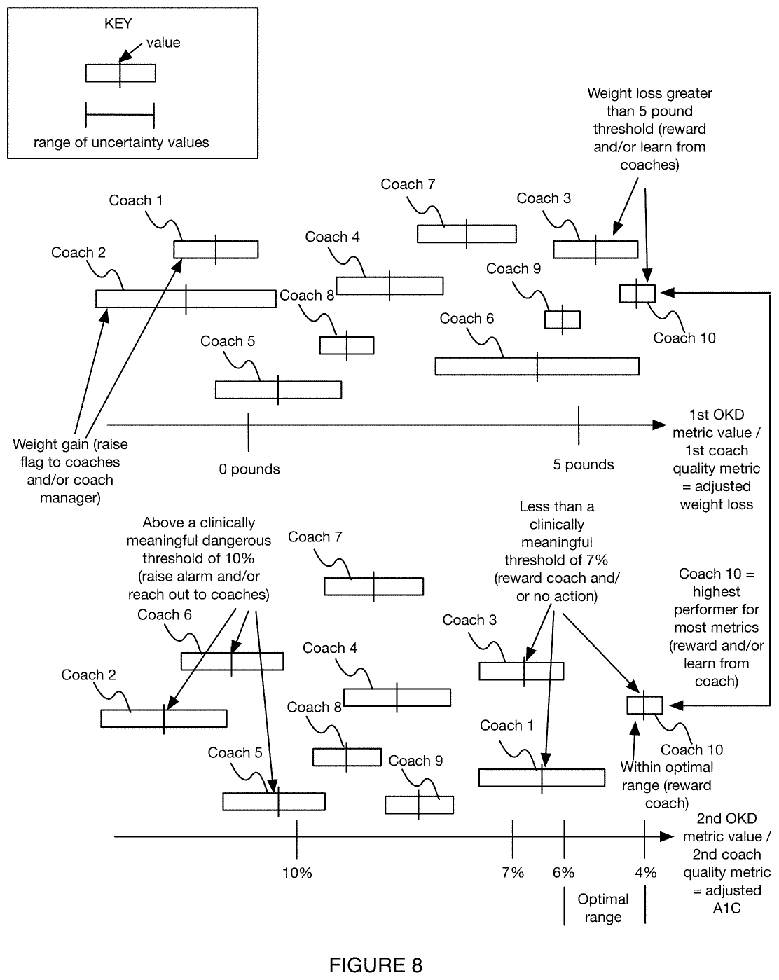

Any or all of the set of OKD metric values can be associated with a range of uncertainty (e.g., set of uncertainty values as shown in FIG. 7, as shown in FIG. 8, etc.), such as those resulting from the implementation of the model (e.g., Bayesian hierarchical model). A large uncertainty value can signal, for instance, that a particular coach is inexperienced and not associated with a large amount of participant historical data. As the coach coaches more participants, for instance, this uncertainty range may decrease. Additionally or alternatively, the amount of uncertainty can be caused by and/or attributed to any other factors (e.g., variability in the participant cohort, new types of participants in the participant cohort, etc.). Further additionally or alternatively, the OKD metric values can be absent of uncertainty.

Additionally or alternatively, the participant metrics can include (and/or an OKD can be determined based on) any or all of: a participant engagement with the coaching platform (e.g., how often the participant does a weigh-in, how often the participant tracks meals, how often the participant communicates with his or her coach, how much the user trusts his or her coach, etc.); a participant satisfaction level with the coaching platform (e.g., as assessed through one or more surveys); and/or any other suitable metrics.

The metrics are preferably calculated based on a set of models and/or a set of algorithms (e.g., as described above), but can additionally or alternatively be determined in any other suitable ways.

In a preferred variation of S250, S250 includes determining a set of OKDs for the set of participants associated with a coach, wherein the set of OKDs are determined with one or more models as described above. In specific examples, for instance, a hierarchical model (e.g., Bayesian hierarchical model) is used to determine an OKD in the form of an adjusted metric (e.g., adjusted weight loss), wherein the adjusted metric is determined relative to an actual outcome (e.g., actual weight loss) of the participant.

Additionally or alternatively, one or more OKDs can be determined for each participant and aggregated to determine OKDs associated with each coach and subsequently a coach quality in S260.

### 4.6 Method—Determining a Coach Quality Based on the Set of Metrics S260

The method 200 can include determining a coach quality based on the set of metrics S260, which functions to determine an accurate assessment of the coach's performance in coaching a diverse group of participants.

The coach quality is preferably represented in the form of a coach quality metric (e.g., score, quantifiable value, etc.), which can be used in any or all of: comparing a coach's current quality metric with a previous quality metric to determine a coach's progress or decline; comparing a coach's quality metric with one or more thresholds (e.g., to compare the coach's quality with a baseline required coach quality, to categorize a coach's quality metric [e.g., as low quality, medium quality, or high quality], to trigger an action, etc.); comparing a coach's quality metric with another coach's quality metric or an aggregated set of coach quality metrics (e.g., to identify top-performing coaches); and/or to perform any other suitable function. Additionally or alternatively, the coach quality can be represented in the form of any or all of: a rating and/or categorization of a coach (e.g., good vs. neutral vs. bad, expert vs. intermediate vs. novice, satisfactory vs. needs improvement, etc.), a level of progression of a coach (e.g., 25% of the way to expert level coach, 50% of the way to expert level of coach, etc.), a particular aspect of coach quality which the coach excels in and/or could use improvement in (e.g., communication, message content, response time, recommendations, etc.), and/or any other features.

The coach quality is preferably determined based on a set of one or more OKDs associated with the coach's set of participants. In some variations, for instance, a coach quality (e.g., coach quality metric) is determined based on adjusted weight loss metrics calculated for each of the coach's participants. In specific examples, the coach quality is determined based on an average adjusted weight loss per participant. Additionally or alternatively, the coach quality can be determined based on any other OKDs.

In some variations, the coach quality metric is equal to one or more OKD metric values associated with the participants of the coach. In specific examples, for instance, the coach quality metric is equal to the adjusted weight loss associated with his or her cohort of participants. In specific examples in which a coach coaches participants with various OKDs (e.g., adjusted weight loss, adjusted A1C, adjusted blood pressure, as shown in FIG. 8, etc.), the coach quality metric can include multiple metrics, such as one per OKD. Alternatively, the metrics can be aggregated.

Additionally or alternatively the coach quality metric can be determined based on (but not equal to) the OKD metric value(s), such as based on further processing (e.g., normalization, passing through a set of filters, comparison with a lookup table, with a decision tree, etc.).

Further additionally or alternatively, the coach quality can be determined based on any other metrics, any other inputs described above, and/or any other suitable information. In some variations, for instance, coach experience (e.g., number of years of experience coaching, number of participants coached, etc.), coach milestones (e.g., number of participants who have achieved their goals, number of participants who have not dropped out of program, number of participants who have lessened the severity of a medical condition, etc.), and/or any other coach information. In some variations, additional or alternative to other variations, information associated with participants is factored into the determination of coach quality. The participant information can include, for instance, characteristics associated with the participants (e.g., individually, collectively, etc.), which may indicate a complexity (e.g., difficulty, challenge, etc.) of coaching the participant(s). Examples of this include labels (e.g., critical vs. non-critical, diseased vs. healthy, etc.) associated with the participants, information associated with the participant (e.g., messaging frequency, associated medical conditions, etc.), historical information associated with the participants (e.g., prior history of dropping out of the coaching program), and/or any other suitable information.

The coach quality can optionally include any number of short-term metrics, such as average response time to a participant and/or any other short-term metrics. These are preferably provided as supplementary information (e.g., in a report to a manager of the coach), but can additionally or alternatively be combined with OKD metrics and/or any other metrics (e.g., into an aggregated metric).

The coach quality can be determined based on any or all of: a set of models (e.g., as described above, different than those described above, etc.), equations, algorithms, lookup tables, databases, and/or through any other suitable processes.

Determining (e.g., calculating, approximating, predicting, selecting, etc.) the coach quality can further include analyzing a coach quality metric through any or all of: comparing the coach quality metric with a set of thresholds and/or targets (e.g., coach expectation thresholds, a response time threshold, a threshold amount of time to spend weekly at the coach dashboard, etc.), aggregating one or more metrics, comparing any or all of the metrics with other metrics (e.g., historical metrics, aggregated metrics, etc.), and/or any other suitable processes.

In a first set of variations, the coach quality is determined directly based on one or more OKDs calculated for each of the participants in the coach's cohort. Additionally, the coach quality can be determined based on one or more metrics associated with coach dashboard activity (e.g., time spent on dashboard, response time, etc.) and/or any other suitable metrics.

In a second set of variations, the coach quality is determined based on aggregating multiple OKD metrics from the cohort of participants, wherein the OKD metrics can be of the same type, different types, or any combination.

In a third set of variations, a single OKD (e.g., of each type) is determined for all of a coach's participants collectively.

Additionally or alternatively, a coach quality can be otherwise determined.

4.7 Method—Producing an Output and/or Triggering an Action Based on the Coach Quality S270

The method 200 can include producing an output based on the coach quality S270, which can function to perform any or all of: triggering one or more actions commensurate with coach quality (e.g., rewarding a high quality coach, highlighting an area of improvement to a low quality coach, incentivizing a coach, etc.), maintaining a high quality coach cohort, learning from high performing coaches, directing a coach manager's attention to a subset of struggling or low-performing coaches, updating one or more models, and/or performing any other suitable functions.

S270 is preferably performed in response to S260, but can additionally or alternatively be performed in response to any process(es) of the method, prior to any process(es) of the method, and/or at any other suitable times. Additionally or alternatively, S270 can performed at any or all of: continuously, at a predetermined frequency, at a predetermined set of intervals, at a random set of intervals, in response to a trigger (e.g., by a coach, by a coach's manager, by a participant, etc.), and/or at any other suitable times.

The outputs can include any or all of: an update to a dashboard (e.g., updated coach rating on a coach dashboard and/or participant dashboard), a notification to the coach (e.g., message congratulating him or her, suggesting an area of improvement, etc.), a notification to a coach supervisor, a salary change and/or any other reward (e.g., cash bonus), an addition of participants to the coach's cohort, a removal of participants in the coach's cohort, the production of a report (e.g., to the coach, to a coach supervisor, etc.), an update to the coach's set of responsibilities, an update to the coach's performance requirements (e.g., decreased time to respond to participants, increased time to respond to participants, etc.), and/or any other suitable output(s).

Any or all of the outputs can be automatically triggered, manually triggered (e.g., by a coach supervisor), and/or any combination of both.

In some variations, a high coach quality (e.g., metric above a predetermined threshold, within one or more predetermined categories, above a predetermined percentile of coach qualities, etc.) can trigger any or all of the following outputs: a notification to the coach (e.g., message, text message, email, etc.), a notification to a coach supervisor (equivalently referred to herein as a manager) (e.g., to prompt the supervisor to acknowledge and/or reward the coach), a reward (e.g., salary increase, bonus, etc.) to be sent to the coach, an increase in the number of participants the coach manages, a promotion of the coach (e.g., to a manager, to a higher pay level, etc.), and/or any other suitable outputs.

In some examples, information (e.g., messaging information, coaching style, coaching metrics, caseload, experience level, etc.) associated with coaches having high coach quality metrics, such as consistently high coach quality metrics, can be used to improve a system and/or method for coaching. This can be used, for instance, for any or all of: training one or more coaches (e.g., through inclusion in training materials for new coaches), determining one or more metrics or scores (e.g., any or all of the scores described in U.S. application Ser. No. 17/195,156), training and/or retraining one or more models (e.g., as described in the method 200, other models, models used to determine one or more scores such as a rapport score, etc.), and/or can be used in any other suitable way.

In a specific example, for instance, messaging information associated with a consistently high quality coach (e.g., in an upper decile of all coaches) can be used to train a model used in determining a rapport score of the coaches, wherein the rapport score quantifies a quality of the coach's interactions with his or her participants through messaging.

In some variations, additional or alternative to those above, a low coach quality (e.g., metric below a predetermined threshold, within one or more predetermined categories, below a predetermined percentile of coach qualities, etc.) can trigger any or all of the following outputs: a notification to the coach (e.g., message, text message, email, etc.), a notification to a coach supervisor (e.g., to prompt the supervisor to speak to the coach about areas of improvement, to prompt the supervisor to fire the coach, etc.), a penalty (e.g., salary decrease, suspension in coaching, etc.) to be implemented, a decrease in the number of participants the coach manages, and/or any other suitable outputs.

In some examples, for instance, information associated with coaches having a low quality metric can be used by a manager of the coach to prioritize his efforts toward this coach (rather than coaches who are performing well). In some instances, for instance, this can prompt the manager to take a more detailed look through the coach's messages with participants and/or other detailed information. This can be useful in enabling managers to manage a large number of coaches (e.g., 30, greater than 10, greater than 30, greater than 50, between 20 and 100, etc.), as they can target their more complex coach analyses and/or intense coach monitoring toward only coaches who need it. The managers can prioritize these coaches on their own (e.g., based on the metrics provided such as shown in FIGS. 7-8); additionally or alternatively, this prioritization can be automatically provided to managers (e.g., based on an automated notification sent to the manager, based on an automatically provided prioritized list, based on an automatically gathered set of granular data such as messaging data provided to the manager and associated with low-performing coaches, etc.).

S270 can optionally include aggregating data from multiple coaches and/or multiple coach supervisors. This can be used, for instance to inform and/or update one or more coach training protocols. In a specific example, for example, correlating a drop in quality in an aggregated set of coaches with a particular cause can be used to update coach training to implement these learnings.

S270 can optionally be used in determining an optimal matching between a coach and a participant. In some variations, for instance, when a coach is being selected for a participant, a coach quality (e.g., overall coach quality metric, identified coach strength, etc.) and/or intermediate metrics/parameters (e.g., a medical parameter, demographic information, OKD, coach's prior performance metrics, etc.) can be used to optimally match the participant with a coach.

Additionally or alternatively, S270 can be used in any other suitable ways.

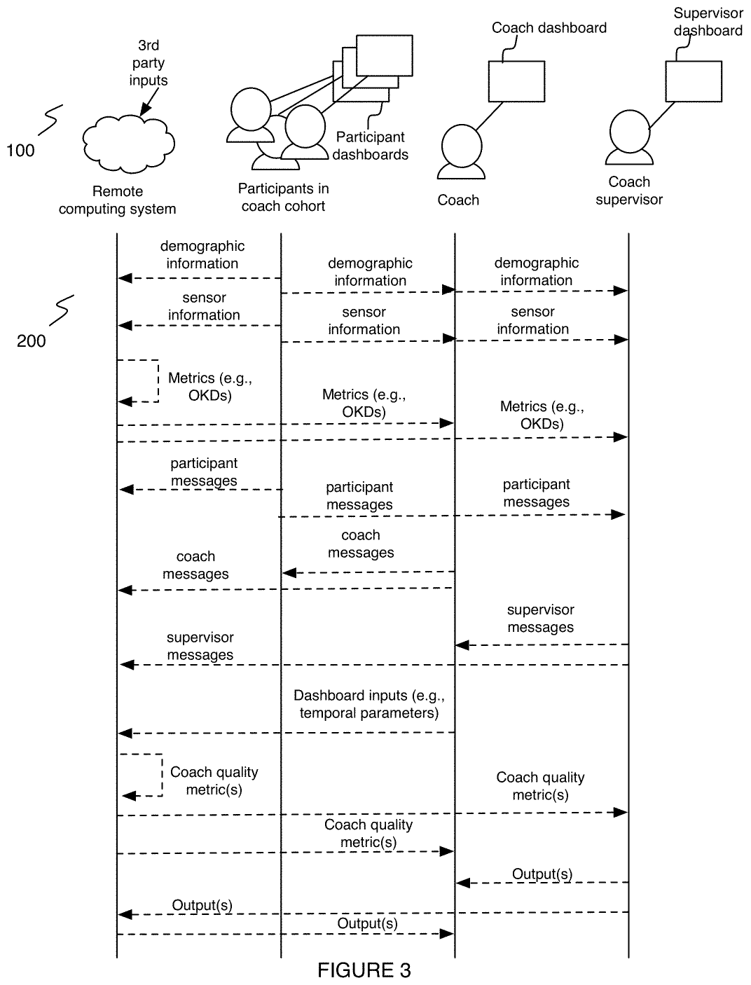

In a variation of the method 200 (e.g., as shown in FIG. 2, as shown in FIG. 3, etc.), the method 200 includes: determining a set of OKDs associated with success of the participant within the coaching platform, wherein the set of OKDs includes an adjusted health outcome metric (e.g., adjusted weight loss metric, adjusted medical parameter metric, etc.) determined through the development and training of a set of models, wherein the set of models determines values for the OKDs based at least in part on participant information (e.g., demographic information); collecting information from each of a set of participants in a coach's participant cohort, wherein the information includes at least participant demographic information and optionally participant sensor information (e.g., weight from a scale); at a computing system (e.g., a remote computing system) determining values of the OKDs for each participant based on the participant information; at the computing system, determining a set of one or more coach quality metrics for the coach based on the OKDs; optionally transmitting the coach quality metrics to a coach supervisor associated with the coach; and optionally producing an output based on the coach quality metric wherein the outputs can be any or all of: determined automatically at the computing system, determined by the coach supervisor based on the coach quality metric, and/or otherwise determined.

Additionally or alternatively, the method 200 can include receiving coach information, such as coach dashboard activity, wherein the coach quality metrics are determined partially or completely by the coach information. In specific examples, for instance, a coach experience level is used to select and/or adjust a model (e.g., hierarchical model) associated with the coach.

In a specific example of the method 200, the method 200 uses a hierarchical model (e.g., Bayesian hierarchical model) to determine one or more OKDs associated with a set of participants, wherein the OKDs are in the form of adjusted parameters. Any or all of the participant inputs are preferably used to cluster and/or identify the participants as belonging to a set of clusters (e.g., income level clusters, gender clusters, etc.), wherein the hierarchical model determines the adjusted parameters based on the cluster information along with actual measurements (e.g., actual weight loss of participant).

Although omitted for conciseness, the preferred embodiments include every combination and permutation of the various system components and the various method processes, wherein the method processes can be performed in any suitable order, sequentially or concurrently.

As a person skilled in the art will recognize from the previous detailed description and from the figures and claims, modifications and changes can be made to the preferred embodiments of the invention without departing from the scope of this invention defined in the following claims.

We claim:

1. A method for automatically determining a quality of a set of coaches in a remote health coaching platform, the method comprising:
for each of the set of coaches:
receiving, at a remote computing system, a set of inputs associated with a set of participants assigned to the coach, wherein the set of inputs comprises, for each participant:
an income level of the participant;
demographic information of the participant; and
a set of measurements;
organizing the set of participants into a set of clusters based on the set of inputs;
with a hierarchical model, automatically determining a set of outcome metrics based on the set of inputs and the set of clusters, wherein the set of outcome metrics comprises an adjusted weight loss for each participant of the set of participants;
automatically determining a coach quality metric based on the set of outcome metrics; and
automatically triggering a set of actions based on the set of coach quality metrics.

2. The method of claim 1, wherein the set of measurements comprises a set of actual weight measurements of the participant.

3. The method of claim 1, wherein automatically triggering the set of actions comprises:
triggering a first action for a first subset of the set of coaches, wherein the first subset is associated with a highest subset of coach quality metrics; and
triggering a second action for a second subset of the set of coaches, wherein the second subset is associated with a lowest subset of the set of coaches.

4. The method of claim 3, wherein the first action comprises updating a second model, wherein the second model is configured to determine a score associated with a messaging style of the coach.

5. The method of claim 4, wherein the score is a rapport score.

6. The method of claim 3, wherein the second action comprises arranging the second subset of coaches at the top of a prioritized list for a manager of the set of coaches.

7. The method of claim 1, wherein the set of inputs further comprises location information associated with the participant.

8. The method of claim 7, further comprising determining a set of social determinants associated with the participant based on the location information, wherein the adjusted weight loss is greater than an actual weight loss of the participant if a score associated with the set of social determinants fall below a predetermined threshold.

9. The method of claim 1, further comprising receiving a set of coach inputs associated with the coach, wherein the set of outcome metrics is further determined based on the set of coach inputs.

10. The method of claim 9, wherein the set of coach inputs comprises at least one of:
coach demographic information;
a level of coach experience; and
an occupational background of the coach.

11. The method of claim 10, wherein a set of parameters of the hierarchical model is determined based on the set of coach inputs

12. The method of claim 11, wherein the hierarchical model is a Bayesian hierarchical model, and wherein the set of parameters comprises a prior of the Bayesian hierarchical model.

13. A method for automatically determining a quality of a set of coaches in a remote health coaching platform, the method comprising:
for each of the set of coaches:
receiving, at a remote computing system, a set of inputs associated with a set of participants assigned to the coach, wherein the set of inputs comprises, for each participant:
an income level of the participant;
demographic information of the participant; and
a set of measurements;
with a set of models, automatically determining a set of outcome metrics based on the set of inputs, wherein the set of outcome metrics comprises an adjusted weight loss of each participant of the set of participants;
automatically determining a coach quality metric based on the set of outcome metrics; and
organizing the set of coaches into a first subset of coaches and a second subset of coaches based on the set of coach quality metrics;
automatically triggering a first action based on the first subset of coaches;
automatically triggering a second action based on the second subset of coaches; and
updating the set of models based on information associated with the first subset of coaches.

14. The method of claim 13, wherein the set of outcome metrics further comprises an adjusted health parameter of the participant.

15. The method of claim 14, wherein the adjusted health parameter comprises at least one of an adjusted A1C level and an adjusted blood pressure.

16. The method of claim 14, wherein the set of coaches is further organized based on the set of outcome metrics.

17. The method of claim 13, wherein updating the set of models comprises updating a model configured to determine a score associated with a messaging style of the coach.

18. The method of claim 17, wherein the information associated with the first subset of coaches comprises a set of messages exchanged between the coach and a set of participants assigned to the coach.

19. The method of claim 17, wherein the second action comprises arranging the second subset of coaches at the top of a prioritized list for a manager of the set of coaches.

20. The method of claim 13, wherein the set of inputs further comprises social determinant information associated with the participant, wherein the adjusted weight loss is greater than an actual weight loss of the participant if a score associated with the social determinant information falls below a predetermined threshold.
